(19) United States
(12) Patent Application Publication
(10) Pub. No.: US 2021/0293775 A1
(43) Pub. Date: Sep. 23, 2021

(54) Analytical Toilet for Assessing Analytes in Excreta

(71) Applicant: Medic, Inc., Provo, UT (US)

(72) Inventors: David R. Hall, Provo, UT (US); K. Jeffrey Campbell, Spanish Fork, UT (US); Anthony E. Pullen, Tucson, AZ (US)

(21) Appl. No.: 17/195,600

(22) Filed: Mar. 8, 2021

Related U.S. Application Data
(60) Provisional application No. 62/986,654, filed on Mar. 7, 2020.

(51) Int. Cl. G01N 33/483; E03D 11/13; A47K 13/24; G16H 10/60; G16H 50/30; G01N 33/68

(52) U.S. Cl. CPC G01N 33/4833; E03D 11/13; A47K 13/24; A61B 10/0038; G16H 50/30; G01N 33/6848; G16H 10/60

(57) ABSTRACT

An analytical toilet is disclosed. The analytical toilet includes a bowl adapted to receive excreta and a sensor comprising a component functionalized to interact with an analyte, such as a biomarker. The analytical toilet presents a sample extracted from urine or feces to the component and the component creates an electronic signal indicating whether or not the analyte is present in the sample.

### CROSS-REFERENCE TO RELATED APPLICATIONS

This application claims priority to U.S. Provisional Patent Application No. 62/986,654 titled “Analytical Toilet for Assessing Analytes Extracted from Feces” filed on 7 Mar. 2020, which is incorporated herein by reference in its entirety.

### TECHNICAL FIELD

The present disclosure relates to analytical toilets. More particularly, it relates to analytical toilets equipped to provide health and wellness information related to excreta deposited by a user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Just a few examples of smart toilets and other bathroom devices can be seen in the following U.S. Patents and Published Applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; US 2018/0165417, entitled “Bathroom Telemedicine Station.” The disclosures of all these patents and applications are incorporated by reference in their entireties.

One particular variety of detection, analysis, and trend tracking is related to biomarkers. “A bio-marker, or biological marker is a measurable indicator of some biological state or condition. Biomarkers are often measured and evaluated to examine normal biological processes, pathogenic processes, or pharmacologic responses to a therapeutic intervention. Biomarkers are used in many scientific fields.” (See https://en.wikipedia.org/wiki/Biomarker). Biomarker information can be a valuable resource in providing for the health and wellness of an individual or population. Some of the uses include being used to detect a disease at its earliest stages and monitoring the progression of key health metrics over time.

Initially, the detection of biomarkers was performed on macro-scale samples, but as technology has improved, equipment both takes up a smaller footprint and is able to use smaller and smaller sample sizes. Additionally, as technology improved, smaller and smaller concentrations of individual biomarkers became detectable. Recent innovation has allowed for the creation of circuitry capable of detecting a variety of desirable, specific, individual molecular elements. Current testing of many biomarkers requires the sample to be processed in a laboratory or clinic. Such testing is also costly, inconvenient and time consuming.

One such example is described in U.S. Pat. No. 7,301,199 “Nanoscale Wires and Related Devices”, which outlines the production of nanometer scale circuitry elements. This technology can be used to make nanoscale versions of numerous components. The '199 patent states “for example, semiconductor materials can be doped to form n-type and p-type semiconductor regions for making a variety of devices such as field effect transistors, bipolar transistors, complementary inverters, tunnel diodes, light emitting diodes, sensors, and the like.” (Page 5, Abstract). The disclosure of the '199 patent is incorporated herein in its entirety.

Additionally, US 2018/0088079 “Nanoscale Wires with External Layers for Sensors and Other Applications” disclosed further details related to producing sensors that use nanoscale wires. For example, it teaches “Certain aspects of the invention are generally directed to polymer coating on nanoscale wires that can be used to increase sensitivity to analytes” (Page 1, Abstract). The disclosure of US 2018/0088079 is incorporated herein in its entirety.

### SUMMARY

In a first aspect, the disclosure provides an analytical toilet comprising a bowl to receive excreta and a sensor. The sensor includes a component functionalized to bind to an analyte such as a biomarker. The toilet presents to the component a sample extracted from urine or feces. The component creates an electronic signal indicating if the biomarker is present in the sample.

In a second aspect, the disclosure provides additional information related to the component, including the use of nanometer scale technology for the component.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 illustrates an analytical toilet with the lid closed, according to an embodiment of the disclosure.

FIG. 2 illustrates an analytical toilet with lid open, according to an embodiment of the disclosure.

FIG. 3 illustrates an analytical toilet with lid closed and a portion of the exterior shell removed, according to an embodiment of the disclosure.

FIG. 4 further illustrates the interior of the toilet of FIGS. 1-3, according to an embodiment of the disclosure.

FIG. 5 illustrates a modular analytical test device attached to a manifold, according to an embodiment of the disclosure.

FIG. 6 illustrates another embodiment of a modular analytical test device.

FIG. 7 illustrates another embodiment of a modular analytical test device.

FIG. 8 illustrates an analytical test device adapted for use with a microfluidic chip, according to an embodiment of the disclosure.

FIG. 9 illustrates an MFC analytic test device with an optical interface, according to an embodiment of the disclosure.

FIG. 10 illustrates an MFC analytic test device with a set of electrical contacts, according to an embodiment of the disclosure.

FIG. 11 illustrates an alternate configuration for a larger MFC analytic test device with additional standardized areas designated for fluidic, electrical, or optical interconnects, according to an embodiment of the disclosure.

FIG. 12 illustrates a detailed view of an exposure event of a sample extracted from feces to a sensor, according to an embodiment of the disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### DEFINITIONS

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” refers to the portion of the toilet below and around the bowl supporting it.

As used herein, the term “user” refers to any individual who interacts with the toilet and deposits excreta therein.

As used herein, the term “excreta” refers to any substance released from the body including urine, feces, menstrual discharge, and anything contained or excreted therewith.

As used herein, the term “manifold” is intended to have a relatively broad meaning, referring to a device with multiple conduits and valves to controllably distribute fluids, namely water, liquid sample and air.

As used herein, the term “test chamber” is meant to refer broadly to any space adapted to receive a sample for testing, receive any other substances used in a test, and apparatus for conducting a test, including any flow channel for a fluid being tested or used for testing.

As used herein, the term “sensor” is meant to refer to any device for detecting and/or measuring a property of a person or substance regardless of how that property is detected or measured, including the absence of a target molecule or characteristic.

As used herein, the term “microfluidics” is meant to refer to the manipulation of fluids that are contained to small scale, typically sub-millimeter channels. The “micro” used with this term and others in describing this invention is not intended to set a maximum or a minimum size for the channels or volumes.

As used herein, the term “microfluidic chip” is meant to refer to is a set of channels, typically less than 1 mm², that are etched, machined, 3D printed, or molded into a microchip. The micro-channels are used to manipulate microfluidic flows into, within, and out of the microfluidic chip.

As used herein, the term “microfluidic chamber” is meant to refer to a test chamber adapted to receive microfluidic flows and/or a test chamber on a microfluidic chip.

As used herein, the term “lab-on-chip” is meant to refer to a device that integrates one or more laboratory functions or tests on a single integrated circuit. Lab-on-a-chip devices are a subset of microelectromechanical systems (MEMS) and are sometimes called “micro total analysis systems” (μTAS).

As used herein, the term “data connection” and similar terms are meant to refer to any wired or wireless means of transmitting analog or digital data and a data connection may refer to a connection within a toilet system or with devices outside the toilet.

As used herein, “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, “analyte” is meant to refer to a substance whose chemical constituents are being identified and measured.

As used herein, the prefix “nano-” is meant to refer to something in size such that units are often converted to the nano-scale for ease before a value is provided. For example, the dimensions of a molecule may be given in nanometers rather than in meters.

As used herein, “miniaturized electronic system” is meant to refer to an electronic system that uses nanometer scale technology.

As used herein, “FET” is meant to refer to a field effect transistor, which is a device which uses an electric field to control the current flowing through a device. FETs are also known by the name “unipolar transistor”.

As used herein, “bind” and similar variants are meant to refer to the property of facilitating molecular interaction with a molecule, such as interaction with a molecular biomarker.

As used herein, “functionalize” and similar variants are meant to refer to a device, especially a nanometer scale device, the surface of which being configured to interact with a specific analyte, such as a specific biomarker.

As used herein, “genomic derived signal” is meant to refer to a molecule generated by the genome of a cell, bacteria, virus, or other nucleic acid carrier, such as DNA, RNA, microRNA, cell-free (circulating) nucleic acids or those of various immunologically related cells.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, an analyte that “interacts” with a sensor is meant to refer to several ways a component (e.g., receptor) of a sensor can detect the analyte. “Interacting” may include reversible binding of an analyte to a component in a sensor. This may also be referred to as labile binding where the analyte is weakly bound to a component in the sensor and can be removed by a removal treatment such as a flushing or cleaning process. “Interacting” may include irreversible binding of an analyte to a component in a sensor where the binding is a one-time event and the component of the sensor or the entire sensor must be replaced after each use. “Interacting” may include a non-binding event wherein the analyte is in the vicinity of the component of the sensor such that the magnetic, optical or electrical properties of the component are perturbed by the presence of an analyte. For example, this may be caused by negative or positive charges located on the surface of the analyte.

### EXEMPLARY EMBODIMENTS

The present disclosure relates to smart toilets with analytical tools (may also be referred to as an “analytical toilet” or a “health and wellness” toilet) which detects, analyzes, and/or tracks the trends of analytes, such as biomarkers, of a user who deposits excreta into the toilet. More specifically, the toilet receives feces from a user, processes the feces in preparation for analysis, and brings a sample extracted from the feces (including processed feces) into a testing area for detection by nanometer scale circuitry component, FET, optical detector, or other similar testing components. The circuitry component has been functionalized to interact with a specific analyte, such as a biomarker, on a molecular or atomic level. The circuitry component provides a data signal depending on whether the specific analyte is present in the excreta sample in contact with it. After the toilet has finished with the feces, the toilet purges the feces from the toilet in preparation for receiving a new sample of excreta.

In accordance with the present disclosure, an analytical toilet that includes an infrastructure for multiple health and wellness analysis tools is provided. This provides a platform for the development of new analytical tools by interested scientists and companies. Newly developed tests and diagnostic tools may be readily adapted for use in a system having a consistent tool interface.

In various exemplary embodiments, the analytical toilet provides a fluid processing manifold that collects and routes samples from the toilet bowl to various scientific test devices and waste handling portals throughout the device

In various exemplary embodiments, the analytical toilet provides multiple fluid sources via a manifold system. The manifold is adapted to connect to a plurality of analytic test devices adapted to receive fluids from the manifold. The manifold is designed to selectively provide a variety of different fluid flows to the analytical test device. These fluids may include, among others, excreta samples, buffer solutions, reagents, water, cleaners, biomarkers, dilution solutions, calibration solutions, and gases such as air or nitrogen. These fluids may be provided at different pressures and temperatures. The manifold and analytical test device are also adapted to include a fluid drain from the analytical test devices.

In various exemplary embodiments, the manifold system provides a standardized interface for analytical test devices to connect and receive all common supplies (e.g., excreta samples, flush water), data, and power. Common supplies may be supplied from within (e.g., reagents, cleaners) or without (e.g., water) the toilet system. The analytical test devices may be designed to receive some or all of the standardized flows. The analytical test devices may also include storage cells for their own unique supplies (e.g., test reagent).

In various exemplary embodiments, the manifold is adapted to direct fluids from one or more sources to one or more analytical test devices. The manifold and analytical test devices are designed such that analytical test devices can be attached to and detached from the manifold making them interchangeable based on the needs of the user. Different analytical test devices are designed to utilize different test methods and to test excreta samples for different constituents.

In various exemplary embodiments, the smart toilet provides an electrical power connection and a data connection for the analytical test device. In a preferred embodiment, the electrical power and data connections use the same circuit. In various exemplary embodiments, the toilet is provided with pneumatic and/or hydraulic power to accommodate the analytical test devices.

In various exemplary embodiments, the smart toilet platform performs various functions necessary to prepare samples for examination. These functions include, but are not limited to, liquidizing fecal samples, diluting or concentrating samples, large particle filtration, sample agitation, and adding reagents. Miniaturized mechanical emulsification chambers show promise for repeatable and sanitary stool preparation. Stool samples may also be liquefied using acoustic energy and/or pressurized water jets.

In various exemplary embodiments, the smart toilet also provides, among other things, fluid transport, fluid metering, fluid valving, fluid mixing, separation, amplification, storage and release, and incubation. The smart toilet also is equipped to provide cleansers, sanitizers, rinsing, and flushing of all parts of the system to prevent cross-contamination of samples. In some embodiments, the system produces electrolyzed water for cleaning.

In various exemplary embodiments, one layer of the fluidic manifold is dedicated to macro-scale mixing of fluids. Sample, diluents, and reagents are available as inputs to the mixers. The mixing chamber is placed in series with all other scientific test devices, allowing bulk mixed sample to be routed to anywhere from one to all stations (i.e., analytical test device interfaces) for analysis. Mixing may also occur in an analytical test device.

In various exemplary embodiments, samples are filtered for large particulates at the fluid ingress ports of the manifold. The fluid manifold uses a network of horizontal and vertical channels along with simple valves to route prepared stool samples to one of several scientific test devices located on the platform.

In various exemplary embodiments, the manifold is constructed using additive layers, and different layers can be customized for particular applications. Standard ports and layouts are used for interfacing with external components, such as pressure sources and flow sensors. In general, characteristic channel volumes at the bottom of the manifold stack are on the order of milliliters. At the top of the manifold stack is the microfluidic science device, which will interface simultaneously with multiple microfluidic chips using standardized layout and pressure seals.

In various exemplary embodiments, the analytic test devices are designed to perform one or more of a variety of laboratory tests. Any test that could be performed in a medical or laboratory setting may be implemented in an analytical test device. These tests may include measuring pulse, blood pressure, blood oxygenation, electrocardiography, body temperature, body weight, excreta content, excreta weight, excreta volume, excreta temperature, excreta density, excreta flow rate, and other health and wellness indicators.

In various exemplary embodiments, the system is adapted to work with a variety of actuation technologies that may be used in the analytical test devices. The system provides electronic and fluidic interconnects for various actuator technologies and supports OEM equipment. In a preferred embodiment, the system is adapted to work with actuator modules that can be attached to the sample delivery manifold and controlled by a central processor. The system platform supports an inlet and outlet for the pressure transducer that interfaces with the fluidic manifold, and electronic or pneumatic connections where required. The system supports a variety of macro- and microfluidic actuation technologies including, but not limited to, pneumatic driven, mechanical pumps (e.g., peristaltic), on-chip check-valve actuators (e.g., piezo-driven or magnetic), electroosmotic driven flow, vacuum pumps, and capillary or gravity driven flow (i.e., with open channels and vents).

One benefit of the present disclosure is the detection, monitoring, and tracking of a user's biomarkers without having any inconvenience aside from what they would otherwise do using the toilet. Without the present disclosure, among other things, people often have to manually collect samples of feces, use equipment they are less familiar with than a toilet, or wait longer for analysis and results. Each of these things can negatively impact a user's experience and/or the quality or accuracy of the results.

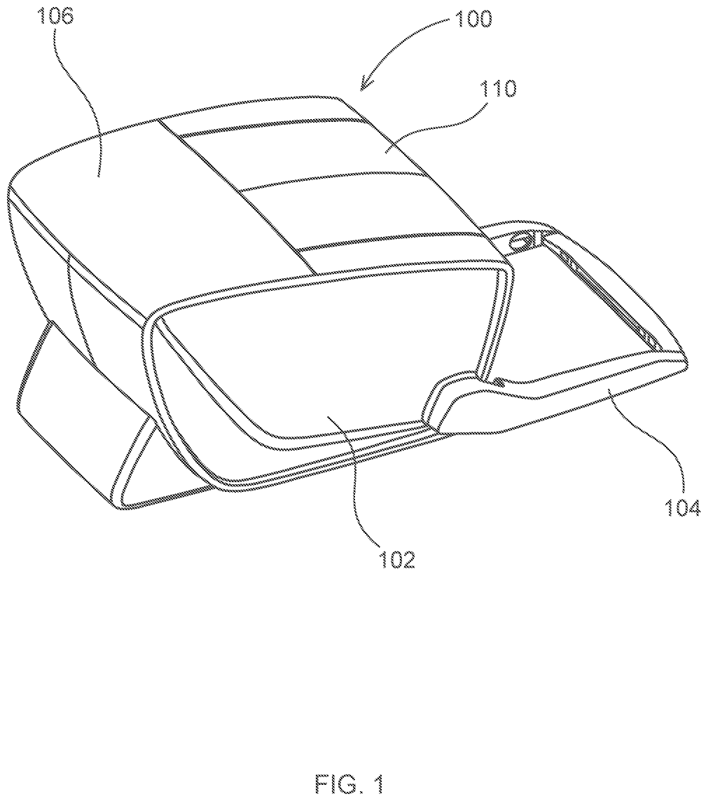

Now referring to FIGS. 1-3, a preferred embodiment of an analytical toilet 100 is shown. FIG. 1 illustrates the analytical toilet 100 with the lid 110 closed, according to an embodiment of the disclosure. FIG. 1 further shows exterior shell 102, foot platform 104 and rear cover 106. The lid 110 is closed to prevent a user from depositing feces in toilet 100 until the toilet is ready for use.

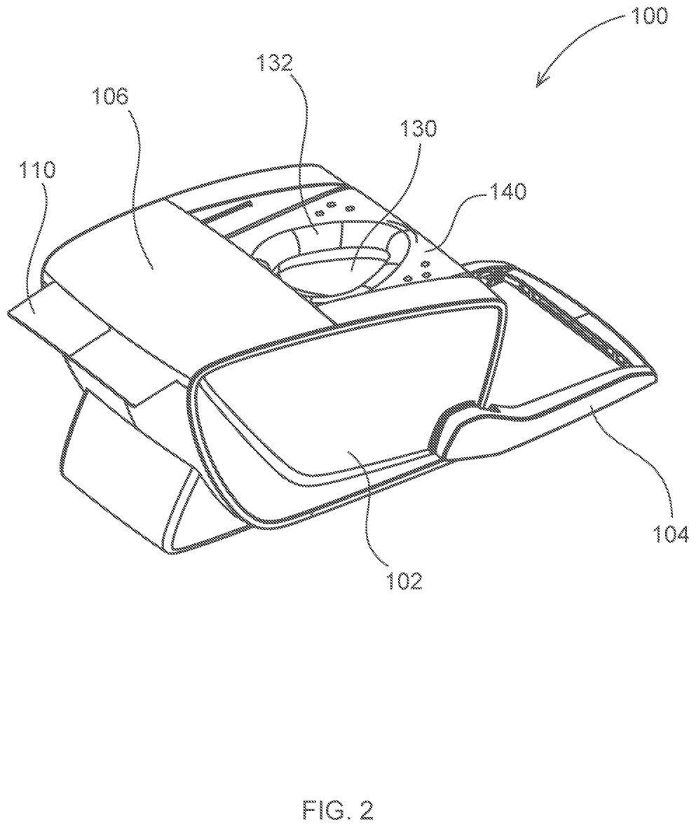

FIG. 2 illustrates toilet 100 with lid 110 open, according to an embodiment of the disclosure. Toilet 100 includes exterior shell 102, rear cover 106, bowl 130, seat 132, lid 110, fluid containers 140 and foot platform 104. Housed within toilet 100 are a variety of features, including equipment, that facilitate receiving excreta, processing feces for analysis, analyzing feces, and disposing of feces. FIG. 2 shows toilet 100 with lid 110 open so a user can sit on seat 132 and deposit feces in toilet 100.

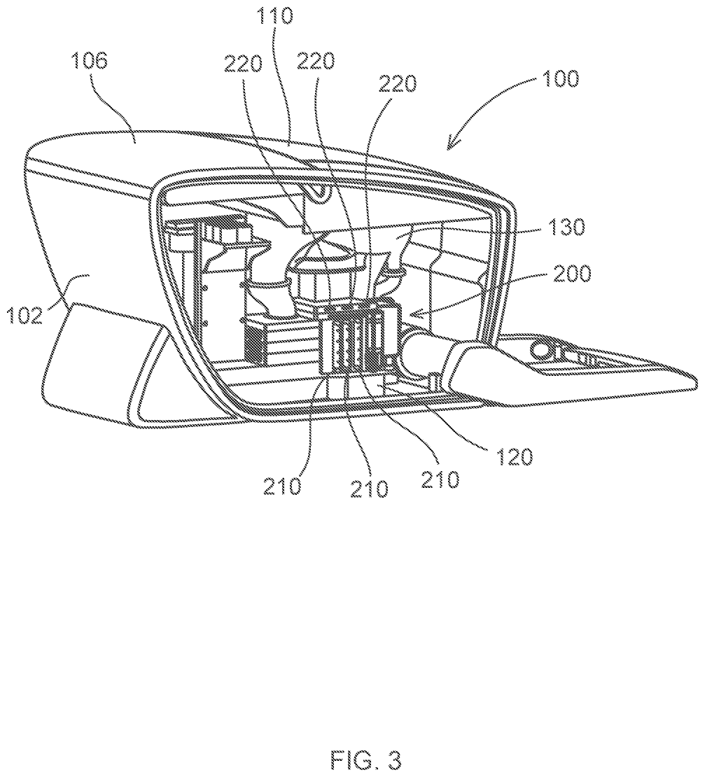

FIG. 3 illustrates toilet 100 with lid 110 closed and a portion of exterior shell 102 removed, according to an embodiment of the disclosure. This allows access to equipment housed within toilet 100. With exterior shell 102 removed, base 120 and manifold area 200 is visible. Manifold area 200 includes test areas 210 and fluidic chip slots 220. Preparation and/or analysis of sample can selectively take place in a test area 210 or fluidic chip slot 220. Manifold area 200 is the area where analysis takes place.

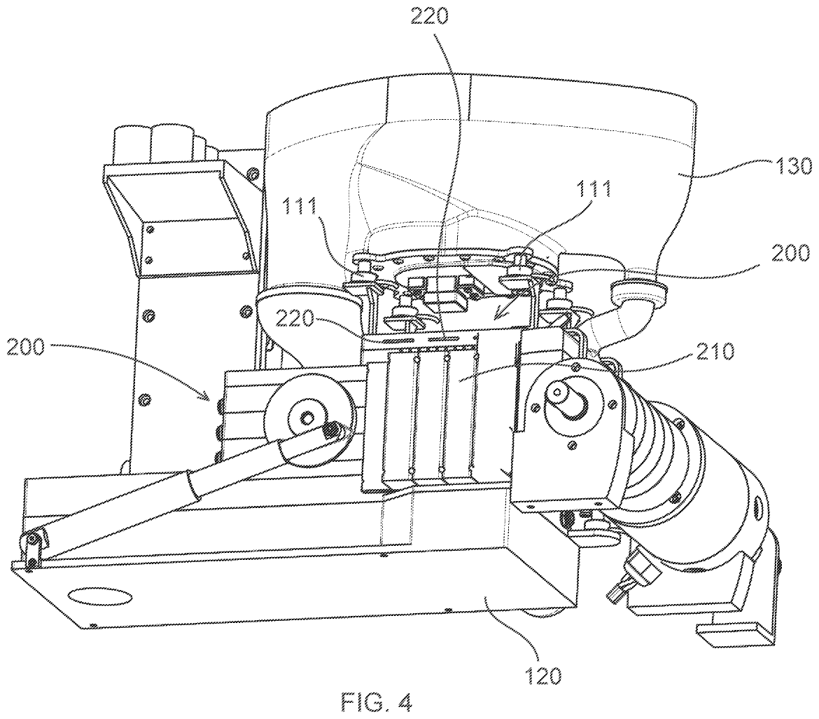

FIG. 4, further illustrates the interior of the toilet of FIGS. 1-3, according to an embodiment of the disclosure. The internal components of the toilet 100 are supported by a base 120. The bowl 130 is supported by one or more load cells 111. A manifold 200 is located below the bowl 130. The manifold 200 comprises a plurality of fluid paths. These fluid paths allow the manifold 200 to move fluids between the bowl 130, fluid containers 140, outside sources (e.g., municipal water supplies), other sources (e.g., air or water electrolyzing unit), analytical test devices 210, and the toilet outlet. The manifold 200 also provides electrical power and data connections to the analytical test devices 210. The manifold 200 can also directly pass fluids and/or solids from the bowl 130 to the toilet outlet.

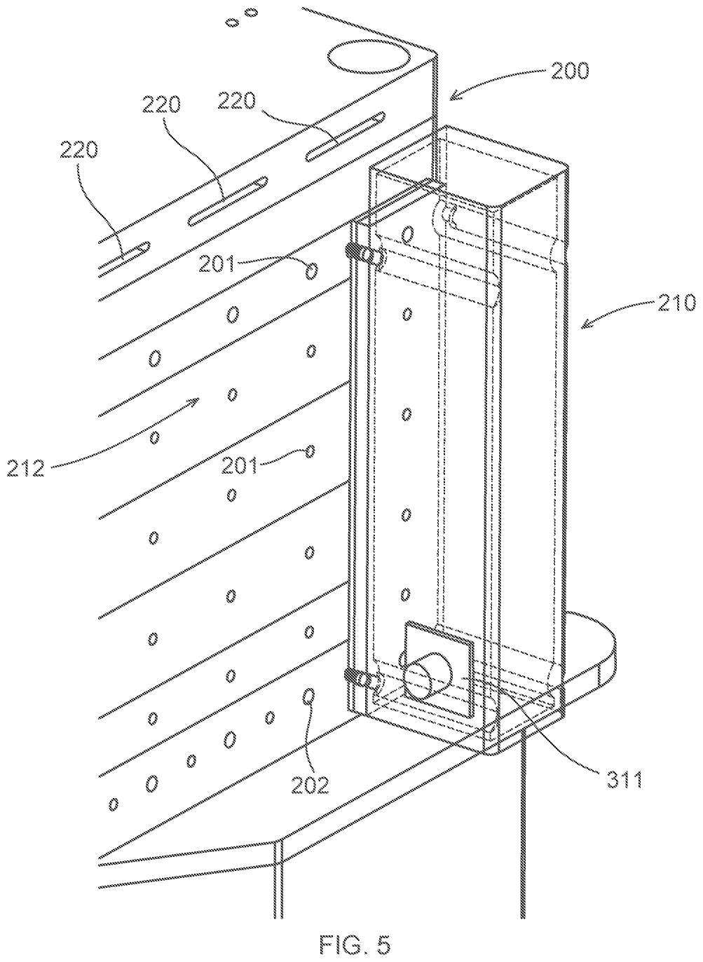

FIG. 5 illustrates a modular analytical test device 210 attached to a manifold 200, according to an embodiment of the disclosure. The manifold 200 is adapted to provide receptacles 210 with standardized connection interfaces for multiple analytical test devices 210. The manifold 200 is shown here with multiple fluid sources 201 for the analytical test device 210. In various embodiments, the manifold 200 may include receptacles 212 for more than one type of analytical test device 210 (e.g., different sizes, fluid supply needs, etc.). Slots 220 are also shown where microfluidic chips (MFCs) that further comprise sensor components may be inserted.

In various exemplary embodiment, the analytical test device 210 includes multiple inlets in fluid communication with the manifold 200. The selected fluid flows are directed into a test chamber with one or more sensors 311 (flow channels internal to the analytical test device not shown in FIG. 5). The sensors 311 may be one or more of electrochemical sensors, spectrometers, chromatography, CCD (charge-coupled device), or metal oxide semiconductor field-effect transistor (MOSFET) including complementary metal oxide semiconductor field-effect transistor (CMOSFET). The analytic test device 210 also includes at least one outlet 202 or drain in fluid communication with the manifold 200.

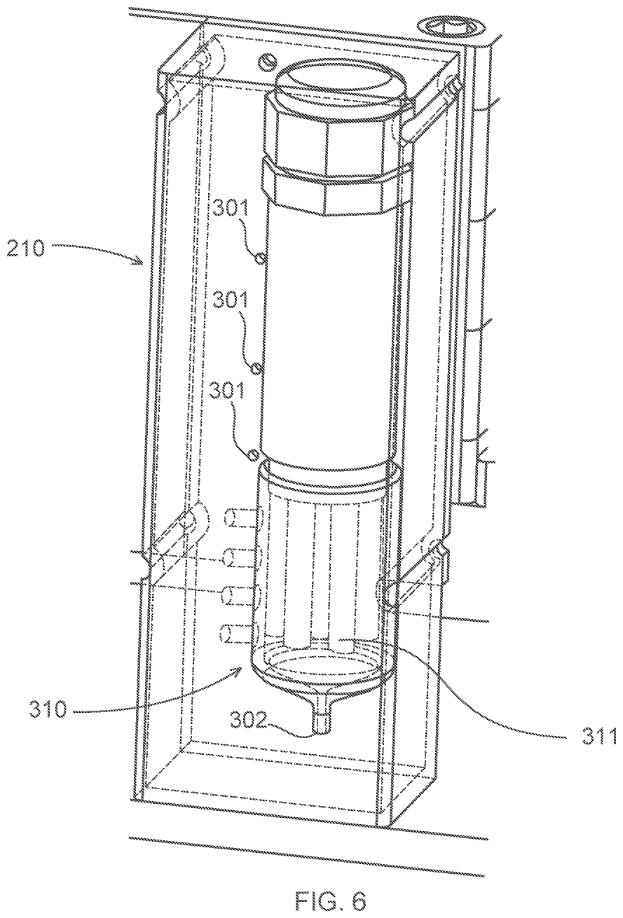

FIG. 6 illustrates another embodiment of a modular analytical test device 210. The analytical test device 210 includes multiple fluid inlets 301, test chamber 310, and at least one fluid outlet 302. The analytic test device 210 includes a test chamber 310 that received fluid flows and contains at least one array of sensors 311.

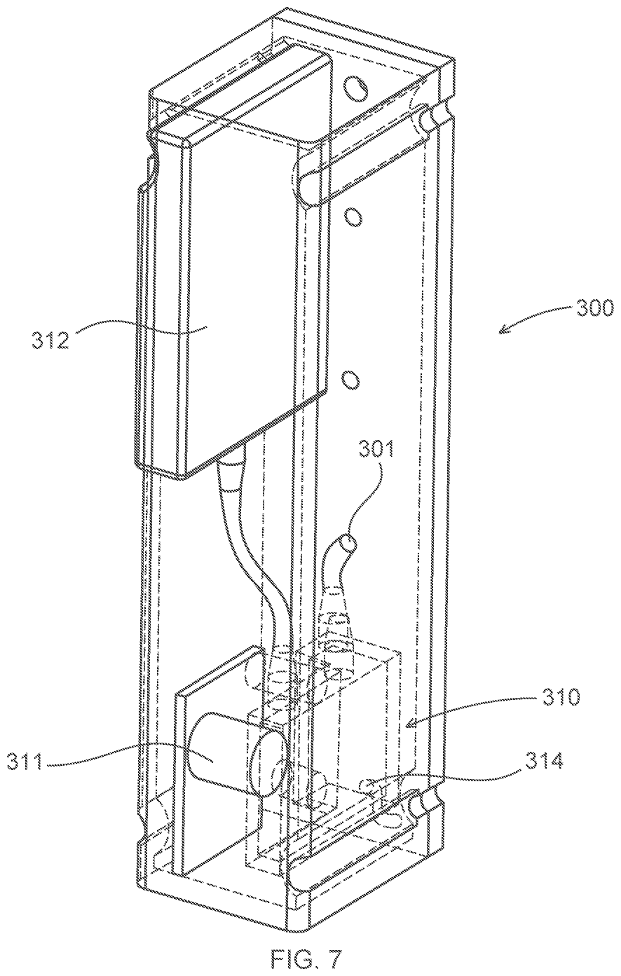

FIG. 7 illustrates another embodiment of a modular analytical test device 210. The analytical test device 210 includes multiple fluid inlets 301, test chamber 310, and at least one fluid outlet (not shown). This embodiment of an analytical test device 210 includes a storage cell 312, also in fluid communication with the test chamber 310. The analytical test device 210 may also include a pump to move fluid (e.g., test reagent) from the cell 312 to the test chamber 310. The analytic test device 210 also includes a camera adjacent to the test chamber 310 to monitor the contents of the test chamber 310. In various embodiments, the test chamber 310 is used to mix a sample extracted from the feces with a reagent that will cause a color change if a target analyte is present in the feces sample. The camera is adapted to detect the color change. In various exemplary embodiments, the camera may be used to observe other characteristics or changes to the sample in the test chamber 310.

There are many ways to incorporate the sensor into the toilet, the selection of which will depend on various factors, including ease of manufacture and maintenance, target market, physical constraints, frequency of use compared to other desired functions of the toilet, and cost. In one preferred embodiment, the sensor is built into a fluidic circuit. More preferably, the fluidic circuit is on a fluidic card. Still more preferably, the fluidic circuit on the fluidic card is a microfluidic circuit on a micro fluidic card. Yet more preferably, the microfluidic circuit interfaces with nano-scale fluidic circuits. Preferably, the fluidic card is inserted into a slot or receptacle of the toilet which connects the fluid circuit on the card to the toilet's fluidic delivery system, enabling the card to receive the sample derived from the feces. Alternatively, the sensor is part of a larger device that may be attached to the toilet, such as a device that processes and/or analyzes the sample extracted from the feces. Alternatively, the sensor is built into the toilet rather than being on a card. Alternatively, the sensor is external to the remainder of the toilet and is connected to receive and/or return fluid from the toilet, such as may be accomplished by connecting the sensor to part of the toilet with tubes or pipes.

### Sample Extraction from Feces

Once feces has been deposited in the toilet, there are many ways it could be processed in preparation for testing and disposal. There are different challenges associated with extracting a sample for testing from feces as opposed to urine. Some pretreatments include a filter, a centrifuge, washing, dilution, or pH normalization. In one preferred embodiment, a portion of feces is separated from urine. The feces is then washed, mixed with buffer, water, and/or a reagent, and presented to the component of a sensor for analysis. Following analysis, the sample is removed from the sensor, and the sensor is cleaned and/or sterilized in preparation for a new sample being presented to the component of the sensor. This method allows for extraction and detection of analytes found on the surface or mixed in with the feces.

In some instances, it may be necessary to use a more aggressive method to extract analytes, such as biomarkers, from feces. The feces may be processed in order to extract a portion of intracellular material contained within intact cells within the feces. This allows for testing of the released genetic material. In particular, this allows for testing of free cell DNA or other intracellular material. The intracellular material may comprise material extracted from bacteria, viruses, yeast, fungi, parasites or a combination thereof. The bacteria, viruses, yeast, fungi, and parasites may be burst or lysed open (also referred to as cytolysis) using a variety of methods. There are several methods that an analytical toilet may be developed to lyse material. Physical methods to lyse cells include mechanical disruption, liquid homogenization, high frequency sound waves, freeze/thaw cycles, and manual grinding. Physical methods tend to be more aggressive which may be necessary to break open fungi or parasites which have more robust and tougher cell material. Physical methods may induce local heating which may lead to protein denaturation and aggregation. The feces may need to be chilled in some instances. Cells burst at different times, so subcellular components may be subjected to ongoing disruptive forces which may lead to too much cellular component degradation.

In some cases, solution-based cell lysis (also referred to as reagent-based cell lysis) may be preferred to release intracellular material in an analytical toilet. Solution-based cell lysis tends to be a more rapid, gentle, efficient, and reproducible method, and leads to a high protein yield. Solution-based cell lysis can be used to extract total protein or subcellular fractions or organelles from various sample types. Solution-based cell lysis works by disrupting the lipid membrane and/or cell wall. Solution-based cell lysis reagents may include detergents such as CHAPS (3-cholamidopropyl dimethylammonio 1-propanesulfonate) or the Triton-X series of nonionic detergents. Solution-based cell lysis reagents may include mammalian cell lysis reagents. Mammalian cell lysis reagents may include radioimmunoprecipitation assay (RIPA) buffer, immunoprecipitation (IP) or co-immunoprecipitation (CoIP) buffer, Thermo Scientific Pierce IP Lysis Buffer, Thermo Scientific M-PER™ Mammalian Protein Extraction Reagent, and Thermo Scientific T-PER™ Tissue Protein Extraction Reagent. Mammalian cell lysis reagents may include bacterial protein extraction agents such as Thermo Scientific BPER™ Bacterial Protein Extraction Reagent. Solution-based cell lysis reagents may include parasite and yeast cell lysis reagent such as Thermo Scientific Y-PER™ Yeast Protein Extraction Reagent or Thermo Scientific I-PER™ Insect Cell Protein Extraction Reagent.

In one embodiment of a method to detect analytes in feces, a sample of feces may first be rinsed/washed with a fluid. A rinsing/washing step can be used to remove urine. Urine can comprise different analytes than feces that may interfere in testing for analytes from feces. The fluid may be aqueous-based such as deionized water, tap water, distilled water or a buffer. The fluid may be an organic material such as methanol, ethanol, isopropanol or other hydrocarbon. The rinse fluid may then be disposed of down the drain or some other location. In some instances, the rinse fluid may be retained for further testing. The buffer may have a pH in the range of about 1-12. The pH of the buffer will vary depending on the analytes being tested for. For example, for a pH range of about 1-2.2, HCl/KCl may be used as a buffer. For a pH range of about 2.2-3.6, glycine/HCl may be used as a buffer. For a pH range of about 2.2-4.0, potassium hydrogen phthalate/HCl may be used as a buffer. For a pH range of about 3.0-6.2, citric acid/sodium citrate may be used as a buffer. For a pH range of about 3.7-5.6, sodium acetate/acetic acid may be used as a buffer. For a pH range of about 4.1-5.9, potassium hydrogen phthalate/NaOH may be used as a buffer. For a pH range of about 5.5-6.7, 2-(N-morpholino) ethanesulfonic acid (MES) may be used as a buffer. For a pH range of about 5.8-7.2, bis-tris methane (BIS-TRIS) may be used as a buffer. For a pH range of about 5.8-8.0, phosphate buffer (PBS) may be used as a buffer. For a pH range of about 6.0-7.2, N-(2-acetamido) iminodiacetic acid (ADA) may be used as a buffer. For a pH range of about 6.1-7.5, piperazine-N,N′-bis(2-ethanesulfonic acid) (PIPES) or N-2-aminoethanesulfonic acid (ACES) may be used as a buffer. For a pH range of about 6.2-7.6, 3-morpholino-2-hydroxypropanesulfonic acid (MOPSO) may be used as a buffer. For a pH range of about 6.3-9.5, 1,3-bis(tris(hydroxymethyl)methylamino)propane (BTP) may be used as a buffer. For a pH range of about 6.4-7.8, BES may be used as a buffer. For a pH range of about 6.5-7.9, 3-(N-morpholino)propanesulfonic acid (MOPS) may be used as a buffer. For a pH range of about 6.8-8.2, 2-[(2-Hydroxy-1,1-bis(hydroxymethy)ethyl)amino]ethanesulfonic acid (TES) or 4-(2-hydroxyethyl)-1-piperazineethanesulfonic acid (HEPES) may be used as a buffer. For a pH range of about 6.9-8.3, 4-(N-morpholino)butanesulfonic acid (MOBS) may be used as a buffer. For a pH range of about 7.0-8.2, 3-(N,N-Bis[2-hydroxyethyl]amino)-2-hydroxypropanesulfonic add (DIPSO) or 2-Hydroxy-3-[tris(hydroxymethyl)methylamino]-1-propanesulfonic acid (TAPSO) may be used as a buffer. For a pH range of about 7.0-9.0, 2-amino-2-(hydroxymethyl)-1,3-propanediol (Trizma base) may be used as a buffer. For a pH range of about 7.1-8.5, 4-(2-Hydroxyethyl)piperazine-1-(2-hydroxypropanesulfonic acid) Hydrate hydrate (HEPPSO) may be used as a buffer. For a pH range of about 7.2-8.5, piperazine-1,4-bis(2-hydroxypropanesulfonic acid) dihydrate (POPSO) may be used as a buffer. For a pH range of about 7.4-8.8, tricine may be used as a buffer. For a pH range of about 7.5-8.9, diglycine (Gly-Gly) may be used as a buffer. For a pH range of about 7.6-9.0, 2-(bis(2-hydroxyethyl)amino)acetic acid (BICINE) or N-(2-hydroxyethyl)piperazine-N′-(4-butanesulfonic acid) (HEPBS) may be used as a buffer. For a pH range of about 7.7-9.1, [tris(hydroxymethyl)methylamino]propanesulfonic acid (TAPS) may be used as a buffer. For a pH range of about 7.8-9.7, 2-Amino-2-methyl-1,3-propanediol (AMPD) or N-tris(Hydroxymethyl)methyl-4-aminobutanesulfonic acid (TABS) may be used as a buffer. For a pH range of about 8.3-9.7, N-(1,1-Dimethyl-2-hydroxyethyl)-3-amino-2-hydroxypropanesulfonic acid (AMPSO) may be used as a buffer. For a pH range of about 8.6-10.0, N-cyclohexyl-2-aminoethanesulfonic acid (CHES) may be used as a buffer. For a pH range of about 8.6-10.3, N-cyclohexyl-2-hydroxyl-3-aminopropanesulfonic acid (CAPSO) may be used as a buffer. For a pH range of about 9.0-10.5, 2-Amino-2-methyl-1-propanol (AMP) may be used as a buffer. For a pH range of about 9.7-11.1, N-cyclohexyl-3-aminopropanesulfonic acid (CAPS) may be used as a buffer. For a pH range of about 10.0-11.4, 4-(cyclohexylamino)-1-butanesulfonic acid (CABS) may be used as a buffer.

In the instances where the rinse fluid is tested, other additives may be added to the rinse fluid or in combination with one or more buffers in order to preserve any biomarkers. Such additives may be one or more reducing agents to protect the biomarkers from damage due to oxidation. Reducing agents may include dithiothreiotol (DTT), 2βmercaptoethanol (BME) or tris(2-carboxyethyl) phosphine (TCEP) or combinations thereof. Another additive may be a protease inhibitor to inhibit protein degradation such as phenylmethylsulfonyl fluoride (PMSF), 4-(2-aminoethyl) benzenesulfonyl fluoride hydrochloride (AEBSF), ethylenediaminetetraacetic acid (EDTA), ethylene glycol-bis (β-aminoethyl ether)-N,N,N′,N′-tetraacetic acid (EGTA), benzamidine, leupeptin, aprotinin, chymostatin, antipain or combinations thereof. Another additive may be an osmolyte to stabilize the biomarker structure (such as a protein) and improve solubility such as glycerol, detergent or a sugar or a combination thereof. Another additive may be an ionic stabilizer to enhance solubility such as a NaCl, KCl or (NH₄)₂SO₄ or a combination thereof. Another additive may be an α-helix stabilizer trifluoroethanol (TFE), trimethylamine N-oxide (TMAO) or a molecular specific chaperone such as a heat shock protein or ligand.

In one embodiment of a method to analyze feces, a user first deposits feces into an analytical toilet. The analytical toilet removes a portion of the feces for further processing. The portion of feces may be rinsed with one or more fluids described previously. Alternatively, the portion of feces may not be rinsed. The rinsed or non-rinsed feces may first be exposed to a physical or reagent-based lysing method to release the intracellular material located within the cellular material within the feces. Once the feces is treated with a lysing method, the lysed material may then be rinsed with a second round of fluids such as buffers, stabilizer, protease inhibitors or other fluids as previously described herein. This preserves the biomaterial and/or prepares it for detection of analytes, such as biomarkers. The rinsed material may then be filtered and transported using a microfluidic system to one or more sensors to analyze the fluid.

In a preferred embodiment, one or more sensors in an analytical toilet described herein is functionalized to interact with a biomarker and produce a signal based on the presence and/or concentration of the biomarker. Often, this means the sensor is configured to respond to an individual molecule or even a specific molecular element or portion of a biomarker. Biomarkers that work well with this kind of sensor include immunological genomic derived signals, DNA genomic derived signals, RNA genomic derived signals, microRNA genomic derived signals, other genomic derived signals, proteins, carbohydrates, lipids, metabolites, and ionic concentrations. In some preferred embodiments, the component of the sensor amplifies the concentration of the targeted analyte. In other preferred embodiments, the component dilutes the concentration of the targeted analyte. In some preferred embodiments, the concentration is neither amplified nor diluted. Use of one category of tests to detect a particular analyte does not preclude use of another test category to detect or measure the same analyte.

There are a variety of classes of biomarkers extracted from feces that may be detectable. For example, some groupings worth being considered are ionic or electrochemical, immunological, chromogenic, labeling or biotinylation, fluorescent binding, staining reactions, and transfection or genotypic. There are many variations on sensors that detect biomarker molecules or atoms that may be used in a health and wellness analytical toilet described herein. It should be known that although the disclosure herein describes extraction of analytes, such as biomarkers, from feces, detection of analytes may also be carried out from urine simultaneously or in series. Health and wellness analytical toilet embodiments described herein may comprise one or more sensors to detect analytes extracted from feces and one or more sensors to detect analytes found in urine.

### FET-Based Sensors

One exemplary class of sensors are biosensor field-effect transistors (BioFETs). BioFETs are based on metal-oxide-semiconductor field effect transistors (MOSFETs) that are gated by changes in the surface potential induced by the binding of biomolecules. Complimentary metal-oxide-semiconductor field effect transistors (CMOSFETs) may also be used. BioFETs comprise a field effect transistor and a biological recognition element or receptor.

BioFET-based sensors for a health and wellness analytical toilet may comprise one or more nanowires or functionalized nanowires to bind with a biomarker, one or more nanocrystals or functionalized nanocrystals, one or more sheets of graphene or functionalized graphene or a combination thereof. These materials are placed in a manner in the FET to bridge the source and drain electrodes. The BioFET may comprise a semiconductor with a functionalized gate. Other sensors include colorimetric based assays, paper-based analytical devices, a luminescent markers or labels, and a fluorescent or otherwise optically stimulated marker or label.

In some embodiments, nanowires for use in BioFETs may include conducting polymers such as polythiophene, polyaniline, polycarbazole, poly(3,4-ethylenedioxythiophene), polypyrrole, polyphenol or combinations thereof. Nanowires may comprise metals such as germanium, silver, gold, platinum, nickel palladium or combinations thereof. Nanowires may comprise two or more metals in a core-shell like arrangement. The metallic nanowires may comprise a thin oxide surface layer for covalent attachment of biomarker receptors. Nanowires may include inorganic oxide materials such as indium oxide (In₂O₃), indium tin oxide (“ITO”), zinc oxide (ZnO), tin oxide (SnO), titania (TiO₂) or silica (SiO₂). In an exemplary embodiment, the nanowire comprises a non-functionalized or functionalized single walled carbon nanotube (SWCNT) or a non-functionalized or functionalized multi-walled carbon nanotube (MWCNT) or a combination thereof. In a more exemplary embodiment, the nanowire comprises silicon (Si). The Si nanowire may comprise p-type or n-type Si. The Si nanowires may have a diameter of about 2 nm or larger. In other embodiments, the Si nanowires may have a diameter of about 2-100 nm. In an exemplary embodiment, the diameter of the Si nanowire may be in the range of about 2-30 nm. The nanowire used may have an aspect ratio of length to diameter in a range of about 500-1500. The nanocrystals may comprise colloidal metal, such as gold, or quantum dots. The nanocrystals may comprise semiconducting or super paramagnetic metal oxides such as iron oxides. Some variations include multiple sensors per component that detect the same biomarker, diverse concentration strengths of the same biomarker, and combinations of multiple biomarkers in an array or assay panel.

The conducting polymers, nanowires and nanocrystals used in FET-based sensors for use in a health and wellness analytical toilet described herein may be exploited for their optical, magnetic and electrical properties to detect various analytes. Their optical, magnetic and electrical properties may be tuned based on their size, how they are made, composition and how they are functionalized. A variety of transduction methods may be used to convert a binding event of a biomarker to a component in a sensor to a detectable and monitorable digital signal. The digital signal may comprise conductivity, resistance, voltage, conductance, fluorescence, spectroscopic, pH, magnetic changes or a combination thereof. In an exemplary embodiment, conductance or voltage or both the conductance and voltage in a FET-based sensor may be monitored when sensing for a biomarker. The conductance or voltage or both the conductance and voltage may be monitored with respect to time when a biomarker interacts with the sensor.

In some embodiments, conducting polymers, nanowires and nanocrystals used in FET-based sensors for use in a health and wellness analytical toilet described herein may be functionalized with one or more monoclonal antibody receptors. The receptors may be covalently attached. Antibody receptors may be used to detect one or more viruses. Such viruses may include DNA, RNA or reverse transcribing viruses. An individual sensor may comprise only one type of antibody to target and detect a specific virus, such as influenza A, adenovirus, COVID-19 or Ebola. In other embodiments, a sensor may comprise two or more antibodies to target and detect two or more different types of viruses.

In other embodiments, conducting polymers, nanowires and nanocrystals used in FET-based sensors for use in a health and wellness analytical toilet described herein may be functionalized with one or more monoclonal antibodies to detect pathogens that cause diseases such as cancer. Cancerous tumor cells release antigens that can be detected. These antigens may be proteins, peptides or polysaccharides. In an exemplary embodiment, a FET-based sensor in an analytical toilet may comprise one or more antibodies to detect antigens released by cancerous cells. Antigens are biomarkers released by cancerous cells may also be referred to as tumor markers. Such biomarkers may include CA 15-3 from breast cancer cells. Prostate specific antigen (PSA) found in prostate cancer cells. CA-125 antigen biomarker commonly found in ovarian cancer cells. Carcinoembryonic antigen (CEA) found in colorectal cancer cells.

Other biomarkers that are extracted from feces and may be detected by a sensor in an analytical toilet described herein include leucine-rich α-2-glycoprotein (LRG1), isoform-1 of multimerin-1 (MMRN1), S100 calcium-binding protein A8 (S100A8), serpin B3 (SERPINB3) and differentiation-44 antigen (CD44) for cervical cancer. Biomarkers bladder-tumor-associated antigen, nuclear matrix protein 22 (NMP22), Calreticulin, clusterin, systatin B, proepithelin, UHRF1, bladder tumor antigen (BTA), human complement factor H related protein (hCFHrp), nuclear matrix protein 22 (NMP22), angiopoeitin (ANG), apolipoprotein E (APOE), interleukin-2 (IL-2), interleukin-6 (IL-6), interleukin-8 (IL-8), interleukin-10 (IL-10), interleukin-12 (IL-12), interleukin-18 (IL-18), interleukin-1ra (IL-1ra), TNF-α, MMP-9, MMP-10, plasminogen activator inhibitor 1 (PAI-1), Semenogelin-2, Vascular endothelial growth factor (VEGF), Coronin-1A, DJ-1, PARK7, Gamma Synuclein, Apo-A1, UP1, soluble Fas, ORM1, HtrA1, hyaluronidase, CP1, CCL18, BLCA-4 and α-1B-glycoprotein for bladder cancer. Collagen α-1(III) peptide, collagen α-1(I) peptide, TMPRSS2-ERG, alpha-methylacyl-CoA racemase and PCA3 biomarkers for prostate cancer. Cathepsin D, NMP22, creatinine, microalbumin, sodium (Na) and potassium (K) biomarkers for renal cancer. Eosinophil-derived Neurotoxin C-terminal Osteopontin fragments and Bcl-2 biomarkers for ovarian cancer. Matrix metallopeptidase-9 (MMP-9), HER-2 and ADAM12 biomarkers for breast cancer. Cystatin SN biomarker for colorectal cancer. IL-6, MMP9 biomarkers for multiple myeloma. It should be noted that some biomarkers are indicative of more than one type of cancer. It also should be noted that this is not an exhaustive list.

Some biomarkers may be indicative of injury or trauma. For example, a sensor in an analytical toilet described herein may be used to detect at least one biomarker extracted from feces and released as a result of a kidney injury. These include urinary neutrophil gelatinase-associated lipocalin (NGAL), cystatin C (CyC), clusterin (CLU), hepatocyte growth factor (HGF), n-glutathione-S-transferase (n-GST), α-GST, kidney injury molecule-1 (KIM-1), osteopontin (OPN), renal papillary antigen (RPA-1), albumin, β2-microglobulin, trefoil factor-3 or urea. Detection of thrombin is indicative of blood clotting in the kidneys.

A sensor in an analytical toilet described herein may be used to detect at least one biomarker extracted from feces and released as a result of cardiovascular disease. This includes N-terminal pro-BNP (NT-proBNP), C-type natriuretic peptide (CNP), mRNA in urine supernatant (US-mRNA), adrenodoxin (ADX), eosinophil cationic protein (ECP), fetuin B (FETUB), growth differentiation factor 15 (GDF15, guanine deaminase (GUAD) or neurogenic notch homolog protein 1 (NOTCH1).

A sensor in an analytical toilet described herein may be used to detect at least one biomarker extracted from feces and released as a result of liver disease. Detection of creatinine, a protein metabolite, is indicative of whether the liver is functioning properly. It can be used to calibrate other test results, e.g. Albumin. Biliruben is indicative of gall stones, infection, and/or liver malfunction. Urobilinogen is indicative of liver diseases.

A sensor in an analytical toilet described herein may be used to detect at least one biomarker extracted from feces and released as a result of a brain disease. This includes azelaic acid, N-methylnicotinamide, α-hydroxybutyrate, choline, formate, and N-methylnicotinamide, oxaloacetate or acetone biomarkers for bipolar disorder. Taurine, glutamate, N-acetyl glycoprotein, 3-(3-hydroxyphenyl)-3-hydroxypropanoic acid, five-carbon sugars, ribose, fructose, 1,2,3-butanetriol and propylene glycol biomarkers for autism. Glucosamine, glutamic acid, vanilmandelic acid, creatinine, α-ketoglutaric acid (α-KG), citrate, valine and glycine for schizophrenia. Serum albumin, apolipoprotein A-I, heparan sulfate proteoglycans (HSPGs), malonate, N-methylnicotinamide, m-hydroxyphenylacetate, hippuric acid, quinolinic acid or tyrosine biomarkers for depression.

A sensor in an analytical toilet described herein may also be used to detect at least one biomarker extracted from feces and released as a result of various diseases such as fibrinogen for chronic obstructive pulmonary disease (COPD) or galactomannan for invasive aspergillosis.

A sensor in an analytical toilet described herein may be used to detect at least one biomarker extracted from feces and released as a result of celiac disease. This includes EDN/EPX, anti-gliadin Ab and anti-transglutaminase Ab.

A sensor in an analytical toilet described herein may be used to detect at least one biomarker extracted from feces and released as a result of Exocrine pancreatic function such as pancreatic elastase.

A sensor in an analytical toilet described herein may also be used to detect nitrites extracted from feces for the presence of bacterial cystitis. Bacterial cystitis is commonly referred to as urinary tract infection (UTI). A FET-based sensor in an analytical toilet described herein may also be used to detect ketones such as acetoacetate (AcAc), acetate (acetone) or Beta-hydroxybutyrate (BHB) for the presence of diabetic ketoacidosis (DKA).

A sensor in an analytical toilet described herein may also be used to detect E. coli extracted from feces for the presence of a bacterial infection.

A sensor in an analytical toilet described herein may be used to detect at least one biomarker extracted from feces and released as a result of inflammatory bowel syndrome (IBS) or inflammatory bowel disease (IBD) such as Crohn's disease and ulcerative colitis (UC). This includes calprotectin (migration inhibitory factor-related protein MRP 8/14, S100 proteins such as A8/A9/A12), alpha-1-antitrypsin (A1A), calgranulin (A, B and C) lysozyme, secretory IgA, albumin, beta-defensin 2, lactoferrin, fecal blood, high-mobility group box 1 (HMGB1) protein, M2-pyruvate kinase, osteoprotegerin, perinuclear anti-neutrophil cytoplasmic antibodies (pANCA), antiSaccharomyces cervisiae antibodies (pASCA), myeloperoxidase, chitinase 3-like 1, defensins, matrix metalloproteinases and bile acids.

A sensor in an analytical toilet described herein may be used to detect at least one biomarker extracted from feces and released as a result of colorectal cancer, such as M2-pyruvate kinase, Fusobacterium nucleatum (Fn), galectin-3 (Gal-3), Clostridium hathewayi (Ch), Bifidobacterium, Bacteroides clarus, Clostridium, Porphyromonas, Lachnospiraceae and Enterobacteriaceae, the myosin class of proteins such as Myosin-Vb, methylated vimentin (mVim), MLH1, SEPT9, beta glucuronidase, uridine diphosphate-glucuronosyltransferase 1A (UGT1A1), carbohydrate antigen (CA 19-9), carcinoembryonic antigen (CEA), DNA methyltransferase 3b (DNMT3b), microRNAs, miR-17-92 and miR-135, miR-21, miR-106a, miR-96, miR-203, miR-20a, miR-326 and miR-92, miR-320, miR-126, miR-484-5p, miR-143, miR-145, miR-16, miR-125b miR-21, EYA4, BMP3, NDRG4, SLIT2, oxidatively modified DNA bases/nucleosides, haptoglobin or hemoglobin.

A sensor in an analytical toilet described herein may be used to detect at least one biomarker extracted from feces and released as a result of Microvillus inclusion disease (MVID), such as mutations in the MYO5B, STXBP2 and STX3 genes.

A sensor in an analytical toilet described herein may also be used to detect ionic and electrochemical changes by analysis of a sample extracted from feces. The pH level can be useful as abnormal values are indicative of medical issues. Amperometry may be measured via ion selective membranes. Specific biomarkers to look for include calcium, sodium, and potassium.

A component in a sensor in an analytical toilet described herein may be functionalized with peptide nucleic acid (PNA). PNA can be used as a gene sensor. A PNA is a non-charged variant of DNA and has high selectivity toward complementary DNA sequences. A PNA sensor is very sensitive with almost no electrochemical response toward DNA with one base mismatch. A PNA-based sensor may be used for detection of the DNA sequence responsible for sickle cell anemia.

A sensor in an analytical toilet described herein may be able to detect one or more viruses that are extracted from a sample of feces. The detectable viruses may be from the coronavirus class including alphacoronoavirus, betacoronovirus, gammacoronavirus, or deltacoronavirus. More specifically, these viruses may include SARS-CoV-2 (also known as COVID-19) or SARS-CoV. A component in a FET-based sensor may be functionalized with angiotensin converting enzyme 2 (ACE2) antibody as a receptor. The ACE2 receptor interacts with the spike protein on the surface of the SARS-COV-2 virus. HIV p24 antigen may be detected to helps in the detection of human immunodeficiency virus (HIV) disease.

A sensor in an analytical toilet described herein may be able to detect polysaccharides that are extracted from a sample of feces. Detection of C-polysaccharide can be useful in determining pneumonia, respiratory infections, treatment effectiveness.

A sensor in an analytical toilet described herein may be able to detect one or more parasites that are extracted from a sample of feces. Such parasites may include protozoan (i.e., single-celled parasites), such as cryptosporidium, microsporidia, and isospora. The parasites may also include parasitic worms (helminths), such as tapeworms, flukes, Fasciolopsis buski, hookworms, microsporidia, protozoa, Balantidium coli, Dientamoeba fragilis, Encephalitozoon hellem, Necator americanus, heterophyes heterophyes, Metagonimus yokogawai, pinworms, trichinosis worms, Giardia intestinalis, Giardia lamblia, Entamoeba histolytica, Cyclospora cayetanenensis, ascarias lumbricoides, Ancylostoma duodenale, Taenia, Cystoisospora belli, Diphyllobothrium, Hymenolepsis, Echinococcus, Dipylidium, Spirometra, Enterobius vermicularis, and Cryptosporidium.

In an exemplary embodiment, sensors used in a health and wellness analytical toilet described herein are capable of detecting non-biomarker molecules extracted from feces. Such molecules may comprise prescription drugs, recreational drugs or illicit drugs. These may include amphetamines, nicotine, cannabinoids, opioids, cocaine, heroin, ethanol, methanol, pharmaceuticals or other various stimulants or depressants.

In an exemplary embodiment, sensors used in a health and wellness analytical toilet described herein are capable of multiplexed detection. Multiplexed detection is necessary for simultaneous detection of multiple biomarkers such as proteins. This is critical for reliable detection of complex diseases such as cancer. In some embodiments, FETS comprising both n-type and p-type Si nanowires with different receptors within the same sensor may be required for reliable cancer and other disease detection.

In some instances, the high ionic strength environment in a health and wellness analytical toilet from feces may adversely affect the accuracy and precision of the FET-based biomarker sensor. In some embodiments, a biomolecule permeable layer may be located over the sensor. The biomarker permeable layer may be substantially impermeable to ions such that only biomarkers are able to pass through the layer and approach the sensor. The layer may increase the effective Debye screening length in the region immediately adjacent to the sensor surface. This may allow detection of biomolecules in high ionic strength solutions in real-time. In some embodiments, the layer may only be permeable to a target analyte. In some embodiments, the layer may be only permeable to a class of analytes. The layer may be comprised of a membrane. The layer may be porous. The layer may be comprised of a polymer. The polymer may be comprised of polyethylene glycol.

In some embodiments, FET-based sensors in a health and wellness analytical toilet may be combined with other methods of biomarker detection. Additional biomarkers may be measured via a miniaturized mass spectrometer. Alternatively, additional biomarkers may be measured using gas chromatography integrated into the toilet body or positioned adjacent to the toilet. Additional biomarkers may also be measured using fluorescence spectrometry. A fluorescent tag may be covalently or ionically attached to a target molecule. These tags may be a protein, antibody, peptide or amino acid. These tagged molecules may then be used to detect a specific target such as an antigen. In some instances, two or more detection methods, such as those described herein, may be used to detect the same biomarker.

In various exemplary embodiments, microfluidic systems may be used to isolate and transport a sample, add and mix reagents if appropriate, filter out solids, and test the sample for one or more biomarkers on a small scale (i.e., sub-millimeter scale) in a health and wellness analytical toilet described herein. The microfluidic system may comprise an open microfluidic system, continuous-flow microfluidic system, droplet-based microfluidic system, digital microfluidic system, nanofluidic system, paper-based microfluidic system or combinations thereof.

A microfluidic-based biomarker detection system may be located on a microfluidic chip (MFC). In a preferred embodiment, the MFC includes a test chamber with a lab-on-chip (“LoC”) (also known as “test-on-chip”). The LoC may be designed to perform one or more laboratory tests. In various exemplary embodiments, one or more microfluidic chips (MFCs) may be removed or added to the toilet system as desired or needed at any given time, such as for different biomarker tests. In an exemplary embodiment, a DNA microfluidic chip may be used as a component in a biomarker sensor in a health and wellness analytical toilet. The DNA chip may comprise a DNA microarray, such as the GeneChip DNAarray (Affymetrix, Santa Clara, Calif., USA). The DNA microarray comprises one or more pieces of DNA (probes) for biomarker detection. The MFC may comprise one or more affixed proteins in an array-like fashion. In an exemplary embodiment, the proteins are monoclonal antibodies for detection of antigens.

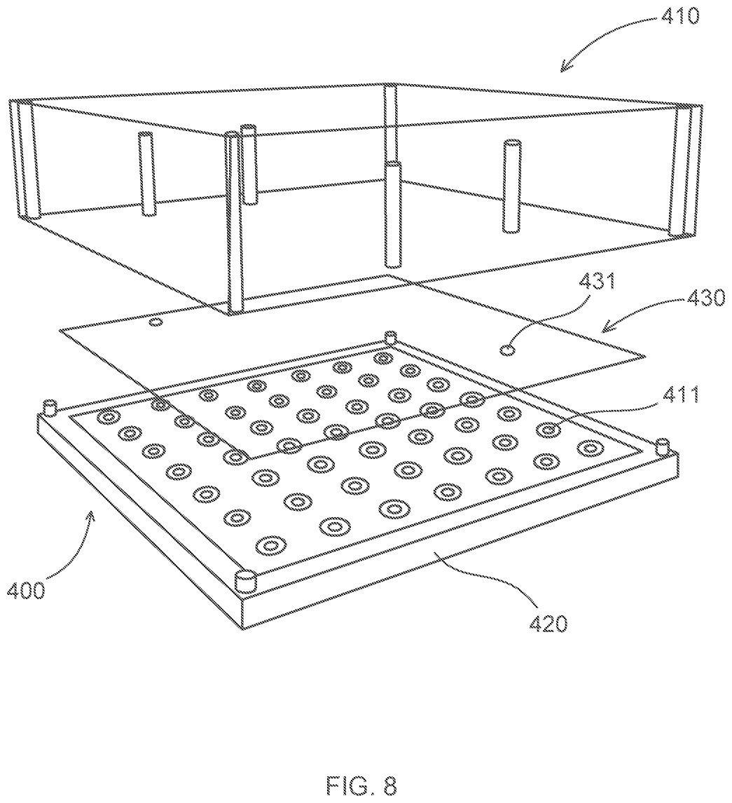

FIG. 8 illustrates an analytical test device 400 adapted for use with a microfluidic chip (MFC), according to an embodiment of the disclosure. The MFC analytical test device 400 includes a test chamber 410 with a lab on chip 411. The LoC 411 may comprise one or more sensor components previously described herein, such as one or more BioFETs. The manifold 200 includes a plurality of slots 220 or other openings for placement of a MFC analytical test device 400. An interface 420 with multiple ports 421, which act as fluid inlets or outlets for the test chamber 410, to provide connections and/or supplies for the MFC analytic test device 400. Protrusions 422 encircle the ports 421 to provide a point of positive contact for the sealing gasket 430, minimizing dead volume. Pores 431 in the gasket 430 select or block possible interactions with the MFC analytical test device 400.

In various exemplary embodiments, the backplate interface 420 is machined or molded with multiple microfluidic pores 421 for ingress of fluids or for removal of fluids. The interface 420 can be used with a variety of MFC analytic test device 400 testing modules. The ports 421 are preferably sealed by placing a gasket 430 between the MFC analytic test device 400 and backplate 420. The gasket 430 may be designed with pores 431 to selectively allow fluid flow through selected ports 421 and block potential flow through others depending on the MFC analytic test device 400 design. The gasket 430 may have alignment holes or features, with matching structures in the interface 420 or MFC analytic test device 400, to facilitate aligning the sheet of material to the pores 431. For example, the gasket 430 may fit snugly in a recess in the interface 420, or alignment pins in the interface 420 may match holes patterned in the gasket 430 by the same process used to create the pores 431. The gasket 430 may have corrugations, or variations in thickness, or be composed of multiple layers of different materials designed to reduce distortions propagating from one point of contact to another, in order to improve the alignment of the gasket 430 to the pores 431 during installation or when the system is pressurized.

In various exemplary embodiments, the gasket 430 may function as a seal for otherwise open channels in the MFC analytic test device 400, permitting pressurized flow in those channels. The gasket 430, typically an electrical insulator, may have electrical contacts built into the top, bottom, or intermediate layers of material, or embedded within the gasket 430 material. The gasket 430 may have electrical contacts built to cause a voltage potential to exist in the fluid. The gasket 430 may have electrical contacts built on the surface to come into contact with the fluid or gas and provide a potential to create electrochemical interactions. The gasket 430 may have electrical contacts built on the surface to come into contact with the fluid or gas and measure the electrical potential or ionic current.

In various exemplary embodiments, when the MFC analytic test device 400 is mechanically clamped to the plate 420 with sufficient pressure, the gasket 230 material creates a high-pressure seal between the MFC analytic test device 400 and the interface 420. Pores 431 in the gasket 430 open a channel between the backplate 420 and the MFC analytic test device 400. The gasket 430 material may be optimized for the application, including selecting chemically inert material. Alternatively, each pore 431 may be circumscribed with an elastomer O-ring that provides a seal under pressure.

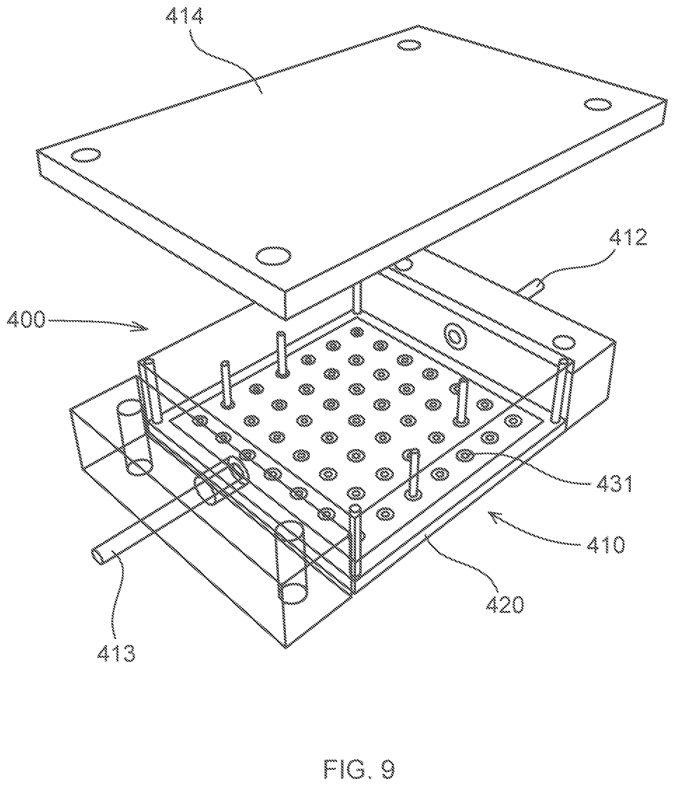

FIG. 9 illustrates an MFC analytic test device 400 with an optical interface, according to an embodiment of the disclosure. In some embodiments, a light source and light detector are connected to the test chamber 410 by fiber optic cables 412 and 413 are part of the test chamber 410 (e.g., spectrometer). The MFC analytical test device 400 is attached to the interface and held in place by a clamp plate. In an alternative embodiment, the light source 412 may be placed in the clamp plate 414 to deliver light to a standardized location normal to the MFC analytic test device 400.

In various exemplary embodiments, the light detector may include various mechanisms for reducing reflections, such as covering the detector with an anti-reflection coating, film. The light detector may be an optical waveguide or other photonic sink.

In various exemplary embodiments, the MFC analytical test device 400 is secured to the interface 420 by a clamp 414. In a preferred embodiment, the clamp 414 serves a dual purpose as a back-side interface for microfluidic ports 421 in the reverse side of the MFC analytic test device 400. Microchannels machined into the clamp 414 in standardized locations may be included in the clamp design.

In various exemplary embodiments, the microfluidic fixtures, including screw-in connectors, may interface with micro-tubing to provide a connection elsewhere in the system, or back to the same chip. The micro-tubing may provide chip-to-chip connections.

In various exemplary embodiments, the clamp may serve as a housing or mounting point for a mirror that directs laser light through the MFC analytic test device 400. Some implementations may use a semi-transparent mirror that allows several MFC analytic test devices 400 arranged in a line to use the same laser beam to interact with MFC analytic test device 400 components. The platform microfluidic interface/manifold provides ports intended for this purpose.

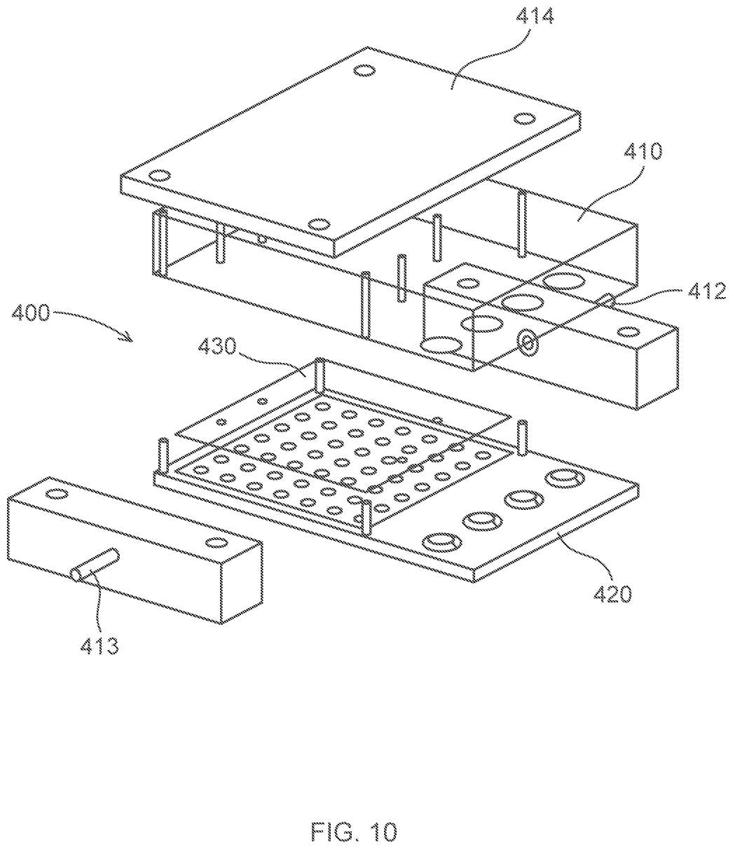

FIG. 10 illustrates an MFC analytic test device 400 with a set of electrical contacts, according to an embodiment of the disclosure. Electrical pads in a standardized location are made available for generic electrical operations, such as providing high and low voltage contacts, control signals, and ground pins. Corresponding pads are also located on the MFC analytic test device 400.

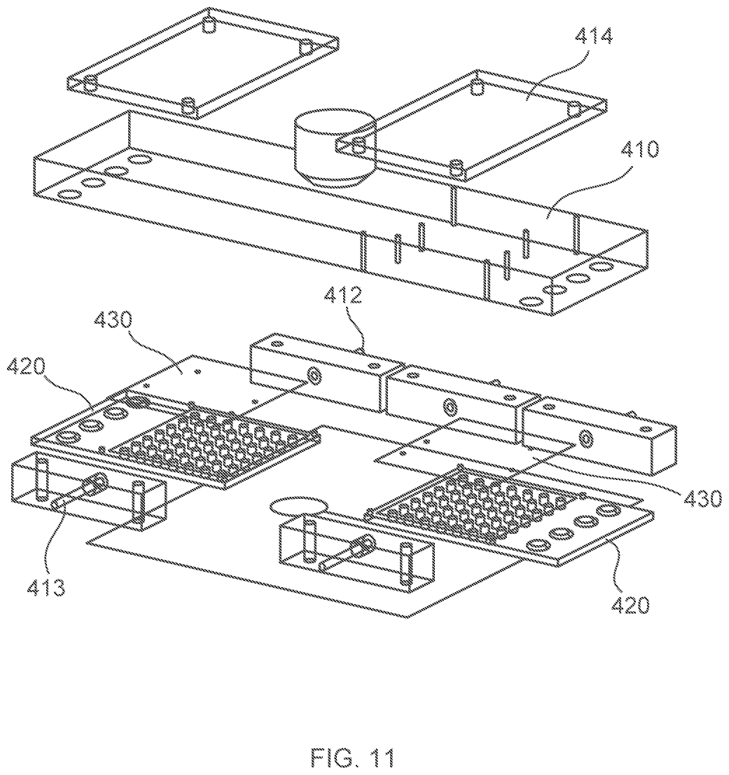

FIG. 11 illustrates an alternate configuration for a larger MFC analytic test device 400 with additional standardized areas designated for fluidic, electrical, or optical interconnects, according to an embodiment of the disclosure. This embodiment shows a designated viewing area, where a light source illuminates the MFC analytic test device 400 from below, and an image detector (possibly including a microscope or other magnifier) examines the output. Other embodiments may include an electronically controlled shutter that selectively blocks the light or selects a pinhole/orifice size for the light. The light source may be visible, UV, or other wavelength range of light emissions. The light source may be wide-band or narrow band.

In various exemplary embodiments, the MFC analytical test device 400 is designed to use very small quantities of reagent. In various exemplary embodiments, reagents are dispensed using technology similar to that used in inkjet printers to dispense ink. In some embodiments, an electrical current is applied piezoelectric crystal causing its shape or size to change forcing a droplet of reagent to be ejected through a nozzle. In some embodiments, an electrical current is applied to a heating element (i.e., resistor) causing reagent to be heated into a tiny gas bubble increasing pressure in the reagent vessel forcing a droplet of reagent to be ejected.

In various exemplary embodiments, the toilet fluidic manifold provides routing. Interconnecting levels of channels allows routing from one port to all others. Each channel includes an accumulator; allows for constant pressure pumping of all active channels simultaneously, while time-multiplexing pump-driven inflow.

In various exemplary embodiments, the manifold has reaction chambers built in for general purpose mixing and filtering operations. Each chamber has a macro-sized channel through which the manifold delivers a sample extracted from feces (filling the reaction chamber), and the chamber has a micro-sized channel. Pumps located internal or external to the manifold drive fluid into the reaction chamber, and into the micro-sized channel. A valve at the output of the macro-channel, and possibly at the output of the micro-channel, controls fluid direction as it exits the reaction chamber.

Microfluidic applications require support infrastructure for sample preparation, sample delivery, consumable storage, consumable delivery or replenishment, and waste extraction. In various exemplary embodiments, the manifold includes integrated support for differential pressure applications, pneumatic operations, sample and additive reservoirs, sample accumulators, external pumps, pneumatic pressure sources, active pump pressure (e.g., peristaltic, check-valve actuators, electro-osmotic, electrophoretic), acoustic or vibrational energy, and light-interaction (e.g., spectrometer, laser, UV, magnification). The acoustic energy source may be a high frequency (54 MHz) bulk acoustic wave (BAW) actuator.

In various exemplary embodiments, the manifold interface has a matrix of ports, possibly laid out in a regular grid. These ports may be activated or closed via an external support manifold. Routing is fully programmable.

In various exemplary embodiments, the manifold directs one or more fluids to the analytical test device 210 or MFC analytical test device 400 to cleanse the devices. These may include cleaning solutions, disinfectants, and flushing fluids. In various exemplary embodiments, the manifold directs hot water or steam to clean sample, reagents, etc. from the devices. In various exemplary embodiments, the toilet systems using oxygenated water, ozonated water, electrolyzed water, which may be generated on an as-needed basis by the toilet system (this may be internal or external to the toilet).

In various exemplary embodiments, waste from the MFCs is managed based on its characteristics and associated legal requirements. Waste that can be safely disposed is discharged into the sewer line. Waste that can be rendered chemically inert (e.g., heat treatment, vaporization, neutralization) is processed and discharged. Waste that cannot be discharged or treated in the toilet system is stored, and sequestered if necessary, for removal and appropriate handling.

In various exemplary embodiments, the manifold creates sequestered zones for each of these waste categories and ensures that all products are properly handled. In various exemplary embodiments, the manifold directs flushing water and/or cleansing fluids to clean the manifold and MFC. In some embodiments, high-pressure fluids are used for cleaning. In such an embodiment, the high-pressure fluids are not used in the MFC. In some embodiments, the MFC is removed from the backplate interface and all ports are part of the high-pressure cleansing and/or rinse.

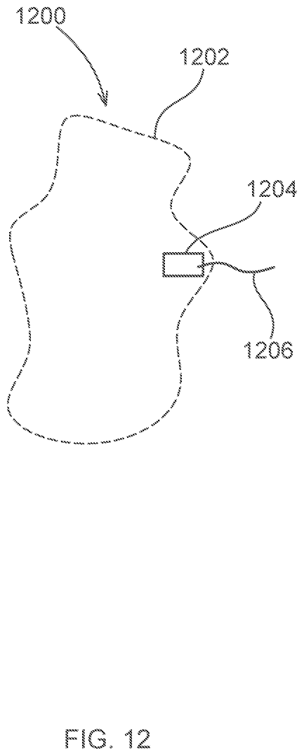

FIG. 12 illustrates a detailed view of an exposure event of a sample extracted from feces to a sensor, according to an embodiment of the disclosure. Exposure event 1200 depicts a sample of excreta 1202 in contact with sensor 1204. Sample 1202 is derived from excreta deposited into the toilet. Sample 1202 may be in a diluted or undiluted state. Depending on the test to be run, the sample may be diluted from about 1:2 to 1:12500, with most tests being run at a dilution of around 1:100.

Sensor 1204 includes a component functionalized to bind with an analyte, such as a biomarker. Alternatively, the component is functionalized to bind with a non-biomarker molecule. Sensor 1204 further includes an electrical lead 1206 that transmits transduction data to a processor.

Following use of the sensor, the toilet may prepare the sensor for future analysis by removing from the test area waste products and other things that might contaminate the next analysis. This could include flushing the sensor, adding a buffer or stabilizing solution, or adding a gas to remove all liquid from the sensor. There are various options to clean, sanitize, and/or prepare the various components of the involved between uses of the toilet. In one preferred embodiment, hot water is run through the fluidic circuit. In another preferred embodiment, oxygenated water is run through the fluidic circuit. In yet another preferred embodiment, a gas is run through the fluidic circuit to remove any liquid from being in contact with the sensor. Alternatively, cleaning and/or preservation agents are run through the fluid circuit. In still another embodiment, if an analyte receptor, such as an antibody receptor, is used in one or more sensors, the sensors are washed with a solution comprising one or more molecules at a predetermined concentration that can interact with and bind with the receptors in a known and predictive manner. This may be necessary when water or other solvent alone may not be sufficient to displace bound analytes, such as biomarkers, in order to clean the sensor. This cleaning method can act as an indicator to show that the sensors are washed and cleared of analytes before the next subject utilizes the toilet. The analytes may be further cleared from the sensor components using a cleaning or preservation agent dispensed from the toilet.

Additionally, temperature can be critical to the preparation, testing, or post processing of the sensor, the fluidic circuit, or the sample. As such, temperature controls may be included to accommodate those need. The controls could be built into the toilet, built into a fluidic circuit, or a result of adding a reagent to the sample. In one preferred embodiment, a resistive wire acts as a heat source to warm the sample and/or the sensor.

### Vision Inspection Sensor

In some embodiments, a vision inspection sensor may be used in a health and wellness analytical toilet to detect abnormalities in a sample of feces. Some vision inspection systems may include KEYENCE (Itasca, Ill., USA), INSPECT.assembly™ (Radiant Vision Systems, Redmond, Wash., USA) or Lake Image Systems (Tring, Hertfordshire, United Kingdom). A vision inspection sensor provides a visual sensor for the presence of parasites, blood, consistency, etc. A vision inspection system may comprise one or more cameras and may provide 2D or 3D images. The inspection system may detect movement, such as from a living parasite.

In various exemplary embodiments, the analytical toilet includes additional health and wellness sensors that may be located in a variety of location. In some embodiments, the seat may contain health and wellness sensors to measure pulse, blood pressure, blood oxygenation, electrocardiography, body temperature, body weight, excreta content, excreta weight, excreta volume, excreta temperature, excreta density, excreta flow rate, and other health and wellness indicators. In a preferred embodiment, the seat is attached to the toilet via a powered quick disconnect system that allows the seat to be interchangeable. This facilitates installing custom seats to include user-specific tests based on known health conditions. It also facilitates installing upgraded seats as sensor technology improves.

In various exemplary embodiments, the lid may contain health and wellness sensors that interact with the user's back or that analyze gases in the bowl after the lid is closed.

In various exemplary embodiments, the analytical toilet includes software and hardware controls that are pre-set so that any manufacturer can configure their devices (i.e., analytical test devices) to work in the system. In a preferred embodiment, the system includes a software stack that allows for data channels to transfer data from the sensors in the medical toilet to cloud data systems. The software and hardware controls and/or software stack may be stored in the analytical toilet or remotely. This would allow scientists to place sensors, reagents, etc. in the system to obtain data for their research. It also allows user data to be individually processed, analyzed, and delivered to the user, or their health care provider, digitally (e.g., on a phone, tablet, or computer application). The seat may also contain sensors to measure fluid levels in the toilet. This could include proximity sensors. Alternatively, tubes in fluid communication with the bowl water could be used to determine changes to bowl fluids (e.g., volume, temperature, rate of changes, etc.).

The toilet disclosed herein has many possible uses, including private and public use. Whether for use by one individual, a small group of known users, or general public use, the toilet can detect, monitor, and create one-time and/or trend data for a variety of analytes, such as biomarkers. This data can be used to prompt a user to seek additional medical, health, or wellness advice or treatment; track or monitor a user or population's known condition; and provide early detection or anticipation of a disease or another condition of which a user or population may wish to be aware.

While the present disclosure often notes the sensor and other equipment supporting feces analysis are located within the toilet, it is possible that some or all of the components are located outside of the toilet. For example, the sample preparation, detection, and processing equipment may be a separate unit adjacent to the toilet which cooperates with the toilet to automatically or semi-automatically receive excreta, prepare a sample of feces for analysis, test the sample, discard the sample, and prevent cross contamination by cleaning and/or sterilizing portions of the toilet and external equipment that do any portion of the described process.

### EXAMPLE

The following example is provided as part of the disclosure as an embodiment of the present invention. As such, none of the information provided below is to be taken as limiting the scope of the invention.

### Example 1. Detecting Calprotectin for Indication of Inflammatory Bowel Disease (IBD)

Example 1 is illustrative of a preferred method of detecting a protein biomarker. The method comprises:
1) A user releases a sample of excreta into an analytical toilet. A 1 cm³ sample of feces is removed and the feces sample is lysed with a pulse from a 54 MHz acoustic energy source.
2) A microfluidic system within the analytical toilet directs, rinses the lysed feces with 5 mL of HEPES buffer solution (>99.5%, 4-(2-Hydroxyethyl)piperazine-1-ethanesulfonic acid, N-(2-Hydroxyethyl)piperazine-N′-(2-ethanesulfonic acid), Millipore Sigma, St. Louis, Mo., USA), filters and transports a 14 sample extracted from the feces to a sensor. A component of the sensor comprises a FET with a silicon nanowire of approximately 10 nm in diameter that bridges the source and drain electrodes. The silicon nanowire is functionalized with the calprotectin specific monoclonal antibody Anti-h Calprotectin 3403 SPTN-5 (Medix Biochemica, Espoo, Finland).
3) The sensor detects a change in conductance in the FET due to an interaction event of a calprotectin protein with the Anti-h Calprotectin 3403 SPTN-5 antibody bound to the nanowire.
4) The sensor relays the computer-readable data to a processor.
5) The processor processes the data and relays the information to the user or a medical professional at an interface.
6) The user or medical professional takes appropriate action in response to the data.
7) The analytical toilet flushes and cleans the sensors and bowl in preparation for the next user.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. An analytical toilet comprising:
a bowl adapted to receive excreta;
a sensor comprising a component configured to interact with an analyte; and
a passage for transferring a sample of excreta to the sensor;
wherein, when the sample is brought into contact with the sensor, the sensor indicates the presence of the analyte by a distinct electric signal.

2. The analytical toilet of claim 1 wherein the excreta is feces.

3. The analytical toilet of claim 2 further comprising a device configured to extract a sample from the feces and introduce it into the passage.

4. The analytical toilet of claim 1, wherein a property of the electric signal is indicative of the concentration of the analyte in the sample.

5. The analytical toilet of claim 1 wherein the component comprises a nanowire Field Effect Transistor with a surface functionalized to bind with the analyte.

6. The analytical toilet of claim 1 wherein the analyte is selected from the group consisting of parasites, fungi, viruses, bacteria, DNA, RNA, proteins, nucleic acids, amino acids, peptides, polysaccharides, salts, metabolites, and fragments thereof.

7. The analytical toilet of claim 1, wherein the sensor is located on a sensor element, wherein the toilet comprises a slot for receiving the sensor element, and wherein the slot provides an interface whereby, when the sensor element is inserted in the slot, the sensor is aligned with the passage and provided with electrical power and data communication.

8. The analytical toilet of claim 1, wherein the sensor can interact with two or more different analytes at the same time.

9. The analytical toilet of claim 1, wherein the component comprises one or more of a semiconductor-based FET with a functionalized gate, a colorimetric based assay, a test strip, a fluorescent or luminescent molecule, and one or more monoclonal antibodies.

10. The analytical toilet of claim 1, wherein the analyte is measured using a miniaturized mass spectrometer.

11. The analytical toilet of claim 1, further comprising an additional sensor that uses gas chromatography.

12. The analytical toilet of claim 1, wherein data from the sensor is used to generate individual trend data over time of the analyte in the excreta of a particular user.

13. The analytical toilet of claim 12, wherein the individual trend data is reported to the user to enhance health and wellness.

14. The analytical toilet analytical toilet of claim 13, wherein the individual trend data from multiple users is combined to generate population trend data.

15. The analytical toilet of claim 1, wherein presence of the analyte is an antecedent to the development of a disease in the user or is indicative of a disease or class of diseases in the user.

16. The analytical toilet of claim 1, wherein data from the sensor is used to monitor for or track a specific health and wellness condition or to forecast the health and wellness condition of a user.

17. The analytical toilet of claim 1, further comprising a layer that is permeable to a biomolecule.

18. The analytical toilet of claim 1 wherein the sensor comprises:
a miniaturized mass spectrometer to detect a biomarker;
wherein, the sensor detects a sample extracted from the feces and the miniaturized mass spectrometer creates an electric signal indicating if the biomarker is present in the sample.

19. The analytical toilet of claim 1 wherein the sensor comprises:
an optical detector to detect a biomarker;
wherein, the sensor detects a sample extracted from the feces and the miniaturized mass spectrometer creates an electric signal indicating if the biomarker is present in the sample.

20. The analytical toilet of claim 1 wherein the sensor comprises:
using gas chromatography to detect a biomarker;
wherein, the sensor detects a sample extracted from the feces and creates an electric signal indicating if the biomarker is present in the sample.

21. The analytical toilet of claim 1 wherein the sensor comprises:
a MOSFET to detect a biomarker;
wherein, the sensor detects a sample extracted from the feces and creates an electric signal indicating if the biomarker is present in the sample.

22. A method to analyze feces comprising:
a bowl receives feces;
a device extracts a sample from the feces;
cellular material within the feces is lysed;
the lysed cellular material is rinsed with a buffer to form a rinse solution; and
the rinse solution is transferred through a passage to one or more sensors comprising a component configured to interact with an analyte;
wherein, when the sample is brought into contact with the sensor, the sensor indicates the presence of the analyte by a distinct electric signal.
